SEQUENCE LISTING (4 sequences)

SEQ ID NO: 1 - DNA, 1362 bp, Homo sapiens
atgggcgtcctgggccgggtcctgctgtggctgcagctctgcgcactgacccaggcggtctccaaactctgggtccccaacacggacttcgacgtcgcagccaactggagccagaaccggaccccgtgcgccggcggcgccgttgagttcccggcggacaagatggtgtcagtcctggtgcaagaaggtcacgccgtctcagacatgctcctgccgctggatggggaactcgtcctggcttcaggagccggattcggcgtctcagacgtgggctcgcacctggactgtggcgcgggcgaacctgccgtcttccgcgactctgaccgcttctcctggcatgacccgcacctgtggcgctctggggacgaggcacctggcctcttcttcgtggacgccgagcgcgtgccctgccgccacgacgacgtcttctttccgcctagtgcctccttccgcgtggggctcggccctggcgctagccccgtgcgtgtccgcagcatctcggctctgggccggacgttcacgcgcgacgaggacctggctgttttcctggcgtcccgcgcgggccgcctacgcttccacgggccgggcgcgctgagcgtgggccccgaggactgcgcggacccgtcgggctgcgtctgcggcaacgcggaggcgcagccgtggatctgcgcggccctgctccagcccctgggcggccgctgtccccaggccgcctgccactttgccctccggccccaggggcagtgctgtgacctctgtggagccgttgtgttgctgacccacggccccgcatttgacctggagcggtaccgggcgcggatactggacaccttcctgggtctgcctcagtaccacgggctgcaggtggccgtgtccaaggtgccacgctcgtcccggctccgtgaggccgatacggagatccaggtggtgctggtggagaatgggcccgagacaggcggagcggggcggctggcccgggccctcctggcggacgtcgccgagaacggcgaggccctcggcgtcctggaggcgaccatgcgggagtcgggcgcacacgtctggggcagctccgcggctgggctggcgggcggcgtggcggctgccgtgctgctggcgctgctggtcctgctggtggcgccgccgctgctgcgccgcgcggggaggctcaggtggaggaggcacgaggcggcggccccggctggagcgcccctcggcttccgcaacccggtgttcgacgtgacggcctccgaggagctgcccctgccgcggcggctcagcctggttccgaaggcggccgcagacagcaccagccacagttacttcgtcaaccctctgttcgccggggccgaggccgaggcctga

SEQ ID NO: 2 - protein, 453 aa, Homo sapiens
MGVLGRVLLWLQLCALTQAVSKLWVPNTDFDVAANWSQNRTPCAGGAVEFPADKMVSVLVQEGHAVSDMLLPLDGELVLASGAGFGVSDVGSHLDCGAGEPAVFRDSDRFSWHDPHLWRSGDEAPGLFFVDAERVPCRHDDVFFPPSASFRVGLGPGASPVRVRSISALGRTFTRDEDLAVFLASRAGRLRFHGPGALSVGPEDCADPSGCVCGNAEAQPWICAALLQPLGGRCPQAACHSALRPQGQCCDLCGAVVLLTHGPAFDLERYRARILDTFLGLPQYHGLQVAVSKVPRSSRLREADTEIQVVLVENGPETGGAGRLARALLADVAENGEALGVLEATMRESGAHVWGSSAAGLAGGVAAAVLLALLVLLVAPPLLRRAGRLRWRRHEAAAPAGAPLGFRNPVFDVTASEELPLPRRLSLVPKAAADSTSHSYFVNPLFAGAEAEA

SEQ ID NO: 3 - DNA, 10872 bp, Homo sapiens
atgatgaacatgtctttaccttttctttggagtttgcttaccttattaatatttgctgaagtaaatggcgaagctggagaacttgagctgcagagacaaaaaagaagcatcaatctccaacagcctcgaatggctacagagagaggaaatttggtgtttcttacggggtctgctcaaaacattgagtttagaaccggatccctgggaaaaattaaattaaatgatgaagatctcagtgagtgtttacatcagatccagaaaaacaaagaagatattatagagttaaaagggagtgcaattggtctgcctcaaaatatatctagtcaaatctatcagcttaattccaagctggtggatcttgagagaaaattccaaggcttgcagcagactgttgacaaaaaggtttgcagcagcaatccttgccagaatggtggaacctgcctcaatctgcatgattcctttttttgtatctgtcccccacagtggaagggtcctctctgctcagctgatgttaacgaatgtgagatttactcaggaacacccttgagctgccagaatggaggcacatgtgttaatacaatgggaagttacagttgtcactgcccacctgagacgtacggaccccagtgtgcatccaaatatgacgactgtgaagggggttctgtggcacgctgtgtccatggcatctgtgaggatttaatgcgagagcaagctggagagcccaagtacagctgcgtctgtgatgctgggtggatgttttcacccaacagccctgcctgcacgctggacagagacgagtgcagcttccagcccgggccttgctccacacttgtgcagtgtttcaacactcaaggctctttctactgtggggcctgtccaacaggctggcaaggcaatggatatatttgcgaagatatcaatgaatgtgagataaataacggcggctgttctgtggctccacccgttgagtgtgtgaatacacctgggtcttcccactgccaggcctgtccaccagggtaccagggtgacggaagagtgtgcacactcacagacatctgctcagtcagtaatggaggctgccacccagatgcctcatgctcctcaactctaggttccttacctctctgcacgtgtctcccgggttatactggaaatggttatgggccaaatggatgtgtgcagctcagtaatatttgcctaagtcacccctgtctaaatggacaatgcatcgacactgtctctggttatttttgtaagtgtgactcaggttggacaggtgtcaactgtacagaaaacatcaatgagtgtttgagcaacccctgtttgaatggaggaacttgtgttgatggcgttgattctttcagttgtgaatgcacacgtctctggactggagctctctgtcaggttcctcagcaagtttgtggagagtccctctcaggaataaatggaagcttcagctacaggagcccggatgttggttatgttcatgatgttaactgcttctgggttatcaaaactgaaatgggaaaggtcctgcgtatcactttcacttttttccggttagaatccatggacaactgtccacacgagtttcttcaggtttatgatggagattcctcttctgcttttcaacttggaagattttgtggctccagcctccctcatgaactcctcagcagtgacaatgctctctattttcatctctattctgaacatttaagaaatgggagaggctttacagtaagatgggaaacacagcaaccagagtgtggaggtatcctgactggtccttacggttctattaagtctccggggtatcctggaaactatcccccaggaagagattgtgtctggattgttgtaactagtcctgacctcctggtaacatttacttttgggaccttgagcctcgagcaccatgatgactgcaacaaagattaccttgagattcgagatggtcctttgtatcaggacccccttcttgggaagttctgcaccactttctctgtcccaccgctccagactactggcccctttgccagaattcacttccattcagactcccagattagtgaccaaggcttccatatcacctacttaacatcaccttcggatctgcgttgtggtgggaactacacggacccagagggtgaactcttcttgcctgagttgtctgggcctttcactcacaccaggcaatgcgtctatatgatgaagcagccccagggagaacaaatacaaatcaacttcacccacgtggagctgcaatgccagagtgacagttctcagaattacattgaggttcgagatggtgaaaccttacttggaaaagtctgtggcaacggaaccatctctcacattaaatccattactaatagtgtctggatcaggtttaaaatagatgcttctgttgaaaaagctagtttcagagctgtttatcaagtcgcttgcggggatgaattaactggagaaggggtcattcgctcgcctttttttcctaacgtgtatcctggagaaagaacctgtaggtggaccatccaccagccccaaagccaagtcattctcctcaacttcactgtctttgaaattggaagttctgcccactgtgaaacagattatgttgagattggtagcagttccattttgggttctcctgaaaataaaaagtattgcggtacagacataccttcatttataacatctgtgtacaattttctttatgtcacattcgtgaaaagttcttctactgaaaaccatggtttcatggctaagttcagtgctgaggatttggcatgtggagaaattcttacagaatcaacagggaccattcaaagtcctggccatccaaatgtctacccccacggtatcaactgtacttggcatatattagtccaacctaatcacctgattcatttaatgttcgaaacatttcatctggagtttcattacaattgcacaaacgactacttggaagtttatgacaccgactctgagacatcccttggaagatactgtggaaagtcgatcccgccatctctcacaagcagtggtaactcattgatgctggtgtttgtgactgactccgacctcgcttatgaaggcttcttaataaactatgaagcaatcagtgcagcaacagcatgtttgcaagactacacagatgatttggggacattcacttctccaaacttccccaataattatcccaacaactgggaatgcatttatcggatcacagtgagaactggccaactgattgcagtgcacttcacaaacttctccttggaggaagccattggaaactattatacagattttctggaaatcagagatggaggctatgaaaaatcaccattgctgggaatattctatggctcaaatctacccccaacaatcatctctcatagtaacaaactatggttaaaatttaagagtgaccaaatagacacaaggtctggattctcagcttactgggatgggtcatcaacaggttgcgggggtaatctcaccacttcaagcggcacgttcatatctcccaactacccgatgccctattaccacagctctgaatgctactggtggttgaaatctagccacggcagcgcatttgaactggaattcaaagactttcacttggagcatcatccaaactgcactttagattacctggctgtatatgatggcccaagtagcaactctcatctgctaactcagctttgtggggatgagaaaccccctcttattcgttctagtggagacagcatgtttataaaactgaggacagatgaaggtcagcaaggacgtggcttcaaggctgaataccggcagacatgtgagaatgtggtaatagtcaatcaaacctatggcatcttagagagtatagggtatccgaatccttattctgaaaatcagcattgcaactggaccatccgggcaacaacaggcaacactgtgaactacacatttttagcatttgacttggaacatcacataaactgctccacagattatttagagctctatgatggaccacggcagatgggacgctactgtggagtagacctgccccctccagggagtactacaagctccaagcttcaagtgctgctccttacagatggggttggccgccgtgagaaaggatttcagatgcagtggtttgtttacggttgtggtggagagctgtctggggccacaggctccttcagcagccccgggttccccaacaggtatccaccaaacaaggagtgtatctggtacattaggacggaccccgggagtagcattcagctcaccatccatgacttcgatgtggagtatcattcaaggtgcaactttgatgtcttggagatctatggaggccccgatttccactctcccagaatagcccaactgtgtacccagagatcacctgagaaccccatgcaggtctccagcactggaaatgagctagcaattcgattcaagaccgacttgtccataaatgggagaggcttcaatgcgtcatggcaagcagtcactggaggttgtggtgggattttccaggctcccagtggagagattcattctccaaattaccccagtccttataggagcaacacagactgttcttgggtcattcgggttgacagaaatcatcgtgttctcttgaacttcactgactttgatcttgaaccacaagactcttgtattatggcatacgatggcttaagctccacaatgtcccgccttgccaggacgtgtggaagggagcagctggctaaccccatcgtctcctcaggaaacagcctcttcttgagatttcagtctggcccttccagacagaacagaggcttccgagctcaattcaggcaagcctgcggaggccacatcctcaccagctcatttgatactgtttcctctccacggttccctgccaattatccaaacaatcagaactgcagctggatcattcaagcgcaacctccattaaatcatatcaccctctcttttacccactttgaacttgaaagaagcacaacgtgtgcacgtgactttgtagaaattttggatggcggccacgaagacgcgcccctccgaggccgttactgtggcaccgacatgccccatcctatcacatccttcagcagcgccctgacgctgagattcgtctctgattctagcatcagtgctgggggtttccacaccacggtcaccgcatcagtgtcggcttgtggtggaacgttctacatggctgaaggcatcttcaacagccctggctacccagacatttatccccctaatgtggaatgtgtctggaacatcgtcagttcccctggcaaccggctccagctgtcttttatatctttccagttggaagactctcaggactgcagcagagattttgtggagatccgtgaaggaaatgccacgggtcacttggtgggacgatactgtggaaactccttccctctcaattattcttccatcgttggacataccctgtgggtcagatttatctcagatggttctggcagcggcacgggcttccaggccacatttatgaagatatttggcaatgataatattgtgggaactcatgggaaagtcgcctctcctttctggcctgaaaactacccacataactccaattaccaatggacagtaaatgtgaatgcatctcacgttgtccatggtagaatcttggagatggacatagaagaaatacaaaactgctattatgacaaattaaggatctatgatgggcctagcattcacgcccgcctaattggagcttactgtggtacccagactgaatctttcagctccactggaaattctttgacatttcatttttactccgactcttcaatctcagggaagggattccttctggagtggtttgcagtggatgcacctgatggtgttttacctaccattgctccaggtgcttgtggtggcttcctgaggacgggagatgcacccgtgtttctcttctccccgggctggcctgacagttacagtaatagagtggactgtacgtggctcatccaggctcccgactctaccgtggaactcaacattctttccctggacattgaatctcaccgaacgtgtgcctatgatagccttgtgatacgagatggagataataacttggcccagcagctagcagttctctgtggcagagagatccctgggcccatccggtctactggagagtacatgttcatccgcttcacctcggactccagtgtaaccagggcaggcttcaatgcatcctttcacaagagctgcggtggatatttgcatgcagacagagggatcatcacgtcccccaagtatccagagacttacccatccaacctcaactgttcttggcacgtcctggtccaaagtggcctgaccattgctgtccattttgaacagcctttccagattccaaatggagattcttcttgcaaccagggggattacttggtgctaagaaatggtcctgatatctgttctccacccttgggaccccctggaggaaatggtcatttttgtggcagtcatgcttcatcaactctgttcacctcggataatcaaatgtttgttcagtttatttctgatcacagtaatgaagggcaaggatttaaaatcaaatatgaggcaaagagtttagcctgtgggggcaacgtctacatccatgatgctgattctgctgggtatgtgacctcccccaaccaccctcataattatcccccgcacgctgattgcatttggatcttagcggctccaccggaaacacgcatacagctgcaatttgaagatcgattcgatattgaagtaacacccaactgtacttccaactaccttgagttgcgggatggagtggattcggatgcaccaatactttccaaattttgtgggacatctttgcccagcagtcagtggtcctcaggagaggttatgtatttgagatttcgatctgacaacagccccacacatgtgggattcaaggccaagtattctatagctcagtgtgggggaagagtaccagggcaaagtggtgttgttgaaagcattggacatccaacacttccatacagagacaacttattctgtgagtggcatctccaggggctctctggacactatctcaccatctcttttgaagactttaaccttcagaattcttctggctgtgaaaaagacttcgtggagatctgggacaatcatacctctggaaacatcttgggcagatactgtggaaacaccattcctgacagcatagacacttctagcaatactgctgtggtcaggtttgtcacagacggctctgtgactgcctcaggattcagactgcgatttgaatccagtatggaagagtgtggtggggatcttcagggctctattggaacatttacttctcccaactacccgaacccaaatcctcatggccggatctgcgagtggagaatcactgccccggagggaaggcggatcaccctaatgtttaacaacctgaggctggccacgcatccgtcctgcaacaatgagcatgtgatagtattcaatggcattagaagtaactcaccccagctagagaaactgtgtagtagtgtgaatgtaagcaatgagattaaatcttcaggaaacacaatgaaagtcatttttttcacggatggatccaggccatatggcggcttcactgcttcctatacctccagtgaagatgcagtgtgtggtgggtctcttccaaatactcctgaaggaaactttacttctcctggctatgacggagtcaggaattactcaagaaacctgaactgcgaatggactctcagcaatccaaatcagggaaattcatccatttccattcactttgaagatttttacctagaaagtcaccaagactgtcaatttgatgtcctcgagtttcgagtgggtgatgctgatgggcccctgatgtggagactttgtggtccttcaaagcctacattgccattggttataccttattctcaggtatggattcactttgtcaccaacgaacgtgtagaacacattggattccatgcaaagtattcctttacagattgtggcggaatacagataggtgacagtggagtgatcacaagccccaactatccaaatgcttatgacagcctgacccactgctcttcgctgttggaggccccacaagggcacaccatcactctcacatttagtgactttgatattgaaccccatacaacttgtgcttgggactctgtcactgtcaggaatggtgggtcccctgaatcacccatcataggacaatactgtggaaattcaaaccccaggacaatacagtcaggttccaatcagctggtcgtgacttttaactcagaccattcattgcaaggtggtggattttatgctacgtggaacacacaaactttaggttgtggtggaatatttcattctgataatggtacaatcagatcccctcactggcctcagaattttcccgaaaacagcagatgttcctggacggccattactcacaaaagtaaacacttggagatcagctttgacaacaacttcctaatccccagcggtgatggacaatgtcagaatagcttcgtgaaggtgtgggcaggaactgaggaggtggacaaagccctgctagccactggctgtgggaacgtggctccgggtcccgttatcacaccaagtaacacattcactgccgtcttccagtctcaggaggcaccagctcagggcttctccgcgtcctttgttagccgatgtggaagtaatttcactggcccttcaggttacatcatttctccaaattacccaaaacaatatgacaacaacatgaattgcacctatgtcatagaggctaatcctctgtcagtggtcctcttgacttttgtgtccttccacttagaagctcgttccgctgtgacgggaagctgtgtcaacgatggcgtgcacattatcagaggttacagcgtcatgtccaccccatttgctactgtgtgtggggatgagatgccagctcccctcaccatcgctgggccggttctgcttaacttctactccaacgagcaaatcacagacttcggattcaagttttcctataggataatctcctgtggtggtgtgttcaatttctcttctggaatcatcacaagtcctgcctattcatacgcagactacccaaatgatatgcactgtctgtataccatcaccgttagtgacgacaaggtgatcgagctcaagttcagtgattttgatgtggttccctccacctcctgctcccatgactacctggcaatttacgatggtgccaataccagcgatccccttcttggcaaattctgcggttccaagcgcccaccaaatgtgaagagcagcaataatagtatgctcctggtgttcaagacagattcatttcagacagcaaaaggctggaagatgtctttccggcagacattggggcctcagcaaggatgtggtggttatctgacaggctcgaataatacctttgcctctcctgattctgattcgaatggaatgtatgacaagaatttaaactgtgtatggatcataattgcacctgtaaacaaagtaattcacctcaccttcaatacatttgctctggaggcagcaagtactaggcaaagatgcctttatgattatgtaaagttatatgatggggatagtgaaaatgcgaacttggctggaacgttttgtggttccacagtacctgctccttttatctcttctggtaacttccttacggttcaattcatcagtgacttaacattagagagggaaggatttaatgctacatacaccatcatggacatgccttgtggtggaacatacaatgcaacttggaccccacaaaatatttcatcacccaattcatcagacccagatgtcccattttccatctgtacttgggtcattgattcccctccgcatcagcaggtcaagataactgtgtgggcattacagctgacctcgcaagactgcacgcagaattacttacagcttcaggactcaccgcagggtcacggaaattcaagatttcagttctgtggcagaaatgcttcggctgtgccagtgttttattcttctatgagtactgcaatggtcattttcaaatctggagttgtaaacagaaactctagaatgagtttcacctatcagattgcagattgcaacagagactatcacaaggcatttggcaacctgagaagccctggatggccagataactacgacaatgacaaggattgcaccgttactctcacagccccccagaaccacaccatttccctcttttttcattcacttggcatcgagaactcagttgaatgcagaaacgatttcttggaggtgagaaatggaagtaacagcaattcaccattactgggcaagtactgtggaactctgctgccaaaccctgtcttctctcaaaataatgaactatacctacgatttaagagtgatagtgtaacttctgatcgtggatatgaaatcatctggacttcatcaccctctggatgtggtggaactctttatggagacagaggctcattcaccagccccggctatccaggcacatacccaaacaacacgtactgcgagtgggtccttgttgctcctgctggaaggcttgtcaccatcaacttctacttcatcagcattgacgatccaggagactgtgtccagaactatctcacactctatgatgggcccaacgccagctctccatcctctggaccatactgcggaggcgacaccagcatagctcccttcgtggcttcctcaaatcaggtcttcataaaatttcatgctgattatgcacggcgtccatccgcattccgattaacttgggacagctaa

SEQ ID NO: 4 - protein, 3623 aa, Homo sapiens
MMNMSLPFLWSLLTLLIFAEVNGEAGELELQRQKRSINLQQPRMATERGNLVFLTGSAQNIEFRTGSLGKIKLNDEDLSECLHQIQKNKEDIIELKGSAIGLPQNISSQIYQLNSKLVDLERKFQGLQQTVDKKVCSSNPCQNGGTCLNLHDSFFCICPPQWKGPLCSADVNECEIYSGTPLSCQNGGTCVNTMGSYSCHCPPETYGPQCASKYDDCEGGSVARCVHGICEDLMREQAGEPKYSCVCDAGWMFSPNSPACTLDRDECSFQPGPCSTLVQCFNTQGSFYCGACPTGWQGNGYICEDINECEINNGGCSVAPPVECVNTPGSSHCQACPPGYQGDGRVCTLTDICSVSNGGCHPDASCSSTLGSLPLCTCLPGYTGNGYGPNGCVQLSNICLSHPCLNGQCIDTVSGYFCKCDSGWTGVNCTENINECLSNPCLNGGTCVDGVDSFSCECTRLWTGALCQVPQQVCGESLSGINGSFSYRSPDVGYVHDVNCFWVIKTEMGKVLRITFTFFRLESMDNCPHEFLQVYDGDSSSAFQLGRFCGSSLPHELLSSDNALYFHLYSEHLRNGRGFTVRWETQQPECGGILTGPYGSIKSPGYPGNYPPGRDCVWIVVTSPDLLVTFTFGTLSLEHHDDCNKDYLEIRDGPLYQDPLLGKFCTTFSVPPLQTTGPFARIHFHSDSQISDQGFHITYLTSPSDLRCGGNYTDPEGELFLPELSGPFTHTRQCVYMMKQPQGEQIQINFTHVELQCQSDSSQNYIEVRDGETLLGKVCGNGTISHIKSITNSVWIRFKIDASVEKASFRAVYQVACGDELTGEGVIRSPFFPNVYPGERTCRWTIHQPQSQVILLNFTVFEIGSSAHCETDYVEIGSSSILGSPENKKYCGTDIPSFITSVYNFLYVTFVKSSSTENHGFMAKFSAEDLACGEILTESTGTIQSPGHPNVYPHGINCTWHILVQPNHLIHLMFETFHLEFHYNCTNDYLEVYDTDSETSLGRYCGKSIPPSLTSSGNSLMLVFVTDSDLAYEGFLINYEAISAATACLQDYTDDLGTFTSPNFPNNYPNNWECIYRITVRTGQLIAVHFTNFSLEEAIGNYYTDFLEIRDGGYEKSPLLGIFYGSNLPPTIISHSNKLWLKFKSDQIDTRSGFSAYWDGSSTGCGGNLTTSSGTFISPNYPMPYYHSSECYWWLKSSHGSAFELEFKDFHLEHHPNCTLDYLAVYDGPSSNSHLLTQLCGDEKPPLIRSSGDSMFIKLRTDEGQQGRGFKAEYRQTCENVVIVNQTYGILESIGYPNPYSENQHCNWTIRATTGNTVNYTFLAFDLEHHINCSTDYLELYDGPRQMGRYCGVDLPPPGSTTSSKLQVLLLTDGVGRREKGFQMQWFVYGCGGELSGATGSFSSPGFPNRYPPNKECIWYIRTDPGSSIQLTIHDFDVEYHSRCNFDVLEIYGGPDFHSPRIAQLCTQRSPENPMQVSSTGNELAIRFKTDLSINGRGFNASWQAVTGGCGGIFQAPSGEIHSPNYPSPYRSNTDCSWVIRVDRNHRVLLNFTDFDLEPQDSCIMAYDGLSSTMSRLARTCGREQLANPIVSSGNSLFLRFQSGPSRQNRGFRAQFRQACGGHILTSSFDTVSSPRFPANYPNNQNCSWIIQAQPPLNHITLSFTHFELERSTTCARDFVEILDGGHEDAPLRGRYCGTDMPHPITSFSSALTLRFVSDSSISAGGFHTTVTASVSACGGTFYMAEGIFNSPGYPDIYPPNVECVWNIVSSPGNRLQLSFISFQLEDSQDCSRDFVEIREGNATGHLVGRYCGNSFPLNYSSIVGHTLWVRFISDGSGSGTGFQATFMKIFGNDNIVGTHGKVASPFWPENYPHNSNYQWTVNVNASHVVHGRILEMDIEEIQNCYYDKLRIYDGPSIHARLIGAYCGTQTESFSSTGNSLTFHFYSDSSISGKGFLLEWFAVDAPDGVLPTIAPGACGGFLRTGDAPVFLFSPGWPDSYSNRVDCTWLIQAPDSTVELNILSLDIESHRTCAYDSLVIRDGDNNLAQQLAVLCGREIPGPIRSTGEYMFIRFTSDSSVTRAGFNASFHKSCGGYLHADRGIITSPKYPETYPSNLNCSWHVLVQSGLTIAVHFEQPFQIPNGDSSCNQGDYLVLRNGPDICSPPLGPPGGNGHFCGSHASSTLFTSDNQMFVQFISDHSNEGQGFKIKYEAKSLACGGNVYIHDADSAGYVTSPNHPHNYPPHADCIWILAAPPETRIQLQFEDRFDIEVTPNCTSNYLELRDGVDSDAPILSKFCGTSLPSSQWSSGEVMYLRFRSDNSPTHVGFKAKYSIAQCGGRVPGQSGVVESIGHPTLPYRDNLFCEWHLQGLSGHYLTISFEDFNLQNSSGCEKDFVEIWDNHTSGNILGRYCGNTIPDSIDTSSNTAVVRFVTDGSVTASGFRLRFESSMEECGGDLQGSIGTFTSPNYPNPNPHGRICEWRITAPEGRRITLMFNNLRLATHPSCNNEHVIVFNGIRSNSPQLEKLCSSVNVSNEIKSSGNTMKVIFFTDGSRPYGGFTASYTSSEDAVCGGSLPNTPEGNFTSPGYDGVRNYSRNLNCEWTLSNPNQGNSSISIHFEDFYLESHQDCQFDVLEFRVGDADGPLMWRLCGPSKPTLPLVIPYSQVWIHFVTNERVEHIGFHAKYSFTDCGGIQIGDSGVITSPNYPNAYDSLTHCSSLLEAPQGHTITLTFSDFDIEPHTTCAWDSVTVRNGGSPESPIIGQYCGNSNPRTIQSGSNQLVVTFNSDHSLQGGGFYATWNTQTLGCGGIFHSDNGTIRSPHWPQNFPENSRCSWTAITHKSKHLEISFDNNFLIPSGDGQCQNSFVKVWAGTEEVDKALLATGCGNVAPGPVITPSNTFTAVFQSQEAPAQGFSASFVSRCGSNFTGPSGYIISPNYPKQYDNNMNCTYVIEANPLSVVLLTFVSFHLEARSAVTGSCVNDGVHIIRGYSVMSTPFATVCGDEMPAPLTIAGPVLLNFYSNEQITDFGFKFSYRIISCGGVFNFSSGIITSPAYSYADYPNDMHCLYTITVSDDKVIELKFSDFDVVPSTSCSHDYLAIYDGANTSDPLLGKFCGSKRPPNVKSSNNSMLLVFKTDSFQTAKGWKMSFRQTLGPQQGCGGYLTGSNNTFASPDSDSNGMYDKNLNCVWIIIAPVNKVIHLTFNTFALEAASTRQRCLYDYVKLYDGDSENANLAGTFCGSTVPAPFISSGNFLTVQFISDLTLEREGFNATYTIMDMPCGGTYNATWTPQNISSPNSSDPDVPFSICTWVIDSPPHQQVKITVWALQLTSQDCTQNYLQLQDSPQGHGNSRFQFCGRNASAVPVFYSSMSTAMVIFKSGVVNRNSRMSFTYQIADCNRDYHKAFGNLRSPGWPDNYDNDKDCTVTLTAPQNHTISLFFHSLGIENSVECRNDFLEVRNGSNSNSPLLGKYCGTLLPNPVFSQNNELYLRFKSDSVTSDRGYEIIWTSSPSGCGGTLYGDRGSFTSPGYPGTYPNNTYCEWVLVAPAGRLVTINFYFISIDDPGDCVQNYLTLYDGPNASSPSSGPYCGGDTSIAPFVASSNQVFIKFHADYARRPSAFRLTWDS

(19) United States
(12) Patent Application Publication
(10) Pub. No.: US 2020/0141955 A1
(43) Pub. Date: May 7, 2020

(54) METHOD FOR DETERMINING VITAMIN B12 UPTAKE

(71) Applicant: Societe des Produits Nestle S.A., Vevey (CH)

(72) Inventors: Jerome Feige, Crissier (CH); Alice Pannerec, Lausanne (CH)

(21) Appl. No.: 16/683,501

(22) Filed: Nov. 14, 2019

Related U.S. Application Data
(62) Division of application No. 15/511,003, filed on Mar. 13, 2017.

(30) Foreign Application Priority Data
Sep. 15, 2014 (EP) 14184779.8

(51) Int. Cl. G01N 33/82; A61K 31/714; G01N 33/68; A23L 33/00; A23L 33/15; A61K 9/00; A61K 35/744; A61K 35/745; A61K 35/747; A61K 36/064

(52) U.S. Cl. CPC G01N 33/82; A61K 31/714; G01N 33/6893; A23L 33/30; A23L 33/15; A61K 9/0053; A61K 2035/11; A61K 35/745; A61K 35/747; A61K 36/064; G01N 2800/06; A23V 2002/00; A61K 35/744

(57) ABSTRACT

A method for determining a level of intestinal uptake of vitamin B12 in a subject can include: determining a level of the cubam complex or a component thereof in an isolated sample from the subject; and comparing the level of the cubam complex or a component thereof to reference levels; wherein the level of the cubam complex or a component thereof compared to the reference levels is indicative of the level intestinal uptake of vitamin B12.

### PRIORITY CLAIMS

This application is a divisional of U.S. application Ser. No. 15/511,003 filed Mar. 13, 2017, which is a National Stage Entry of International Application No. PCT/EP2015/070916 filed Sep. 14, 2015, which claims priority to EP App. No. 14184779.8 filed Sep. 15, 2014. The entire contents of the above-referenced patent applications are hereby expressly incorporated herein by reference.

### SEQUENCE LISTINGS

The instant application contains Sequence Listings which have been filed electronically in ASCII format and are hereby incorporated by reference in its entirety. Said ASCII copy, created on Jan. 10, 2020, is named 3712036-03504_SL.txt and is 52,320 bytes in size.

### FIELD OF THE INVENTION

The present invention relates to the field of nutritional deficiency. In particular, the invention relates to a method for determining intestinal uptake of vitamin B12 in a subject.

### BACKGROUND

Vitamin B12 or cobalamin is a class of cobalt-containing hydrosoluble vitamins which cannot be synthetized by the human body and therefore has to be taken up from food or synthesized by the gut microbiota (Hughes et al.; Ann Clin Biochem, 2013. 50(Pt 4): p. 315-.29). The vitamin B12 pool in the human body is composed of several forms: cyanocobalamin, which is inactive and requires conversion for activity, and methylcobalamin and adenosylcobalamin, which are the metabolically active derivatives of vitamin B12.

After unloading from food matrices in the stomach, vitamin B12 binds to the intrinsic factor (IF) in the small intestine where it is absorbed through a specific heterodimeric transmembrane receptor called cubam, composed of the cubilin (CBN) and amnionless (AMN) proteins. Within the enterocytes, the CBN-AMN-IF-B12 complex is degraded in lysosomes through poorly characterized mechanisms, and free vitamin B12 exits the lysosome via the LMBD1 protein. Vitamin B12 is subsequently transported across the cytoplasm and the basolateral membrane of the enterocyte, and enters the bloodstream through a mechanism that requires at least in part the MRP1 transporter.

Vitamin B12 deficiencies can have either inherited or acquired origins. Congenital inherited deficiencies in vitamin B12 can result from mutations in the intrinsic factor causing hereditary intrinsic factor deficiency, or from mutations m cubilin or amnionless causmg Imerslund-Grasbeck syndrome (or hereditary megalobastic anemia) (Aminoff et al.; Nat Genet, 1999. 21(3): p. 309-13).

In contrast to inherited vitamin B12 deficiencies which manifest clinically within the first years of childhood, acquired vitamin B12 deficiencies manifest much later in life and can have very diverse causes.

Inappropriate nutritional intake of vitamin B12 is one possible cause of vitamin B12 deficiency, especially in vegans who are considered to be at risk because the main sources of dietary vitamin B12 are meat, eggs and dairy products.

Altered vitamin B12 absorption is also an important cause of vitamin B12 deficiency. Pernicious anemia is an autoimmune disease causing vitamin B12 deficiency by destroying the gastric parietal cells which produce intrinsic factor. Gastric dysfunction such as atrophic gastritis, gastric surgery, hypochlorhydria or the use of proton pump inhibitors can also induce low vitamin B12 absorption by failing to produce intrinsic factor and/or to release food-borne vitamin B12 because of inadequate gastric pH. Vitamin B12 malabsorption is also observed in chronic pancreatitis, where low pancreatic enzyme secretion alters haptocorrin degradation, and in Crohn's disease. Low vitamin B12 status can also result as a side effect from chronic treatments with widely prescribed drugs such as 4-aminosalicylic acid, used to treat inflammatory bowel diseases, or metformin, used to treat type 2 diabetes (Kozyraki et al., Biochimie, 2013. 95(5): p. 1002-7).

There is emerging evidence for moderate vitamin B12 deficiencies associating with several chronic diseases, especially those such as cardiovascular diseases, stroke, dementia/cognitive impairment or osteoporosis, which have increased prevalence in elderly populations. Several studies have also linked alterations in vitamin B12 with age-related physical dysfunction. In elderly populations, low muscle strength, low gait speed and functional disabilities have been shown to correlate with either vitamin B12 deficiency, or high levels homocysteine and methyl-malonic acid, two biomarkers of vitamin B12 deficiency in tissues (Hughes et al., as above).

The ability to distinguish whether a vitamin B12 deficiency is caused by an insufficient dietary uptake or by impaired intestinal absorption of vitamin B12 is practically important. Particularly, the use of oral vitamin B12 supplementation in subjects suffering from an impaired intestinal uptake is unlikely to be efficacious in remedying the deficiency. In addition, if a vitamin B12 deficiency is caused by an insufficient dietary uptake, oral supplementation is preferred due to cost and compliance factors, for example.

There is no gold-standard measurement for the diagnosis of vitamin B12 deficiency and this, in combination with the complex aetiology of the deficiency, often results in a late diagnosis.

Total cobalamin levels are the most widely used for first-line evaluation and can be measured in serum/plasma through several validated analytical methods such as the traditional microbiological assays, or more modern competitive binding assays, radioimmunoassays or chemiluminescent assays. However, cobalamin assays do not distinguish between active and inactive forms of vitamin B12 and may be vulnerable to interference resulting in normal values despite severe vitamin B12 deficiency.

Other methods used to assess deficiencies in bioactive vitamin B12 are the measurements of holotranscobalamin (vitamin B12 bound to its bioactive serum transporter transcobalamine II), methyl-malonic acid (MMA), which accumulates with low activity of the vitamin B12-dependent enzyme methylmalonylCoA mutase, and homocysteine, which accumulates with low activity of the vitamin B12-dependent enzyme methionine synthase but can be confounded by folate deficiency. These measurements are usually used in combination as none of the methods has sufficient sensitivity and specificity alone.

There is thus a need for alternative methods and approaches for identifying a possible vitamin B12 deficiency in a subject. In particular, methods which enable identification of a possible vitamin B12 deficiency caused by impaired intestinal absorption rather than insufficient nutritional intake would be advantageous.

### SUMMARY

The present inventors have determined that levels of amnionless increase with age in serum or plasma and that this is correlated with a decrease in circulating levels of total vitamin B12. Further, the present inventors have also determined that components of the cubam complex are detectable in non-intestinal samples, including in serum, and that levels of the cubam complex in the serum increase with age.

Thus, in a first aspect the present invention relates to a method for determining a level of intestinal uptake of vitamin B12 in a subject comprising; determining a level of the cubam complex or a component thereof in an isolated sample from the subject; and comparing the level of the cubam complex or a component thereof to reference levels; wherein the level of the cubam complex or a component thereof compared to the reference levels is indicative of the level intestinal uptake of vitamin B12.

In a second aspect the present invention relates to a method for determining if a subject has impaired intestinal uptake of vitamin B12 comprising; determining a level of the cubam complex or a component thereof in an isolated sample from the subject; and comparing the level of the cubam complex or a component thereof to reference levels; wherein the level of the cubam complex or a component thereof compared to the reference levels is indicative of impaired intestinal uptake of vitamin B12.

A higher level of the cubam complex or a component thereof in the sample from the subject compared to the reference levels may be indicative of a reduced intestinal uptake of vitamin B12.

The component of the cubam complex may be amnionless and/or cubulin or a degradation product thereof. In particular, the component of the cubam complex may be amnionless or a degradation product thereof.

The sample may be a serum or a urine sample. In particular, the sample may be a serum sample.

Detection of the level of the cubam complex or a component thereof may employ flow cytometry, antibody-based arrays, enzyme linked immunosorbent assay (ELISA), non-antibody protein scaffolds, radioimmuno-assay (RIA), western blotting, aptamers or mass spectrometry.

The impaired intestinal uptake of vitamin B12 may be indicative of a vitamin B12 deficiency.

The impaired intestinal uptake of vitamin B12 may be associated with a disease selected from a group of: intrinsic factor deficiency, Imerslund-Grasbeck syndrome, megaloblastic anemia, pernicious anemia, Crohn's disease, inflammatory bowel diseases, type 2 diabetes, cardiovascular diseases, stroke, dementia, cognitive impairment, bypass surgery of part of the digestive tract and osteoporosis.

The impaired intestinal uptake of vitamin B12 may be associated with age-related deficiency in vitamin B12. The impaired intestinal uptake of vitamin B12 may be associated with sarcopenia and/or frailty.

In a further aspect the present invention provides a method for diagnosing a disease selected from a group of: intrinsic factor deficiency, Imerslund-Grasbeck syndrome, megaloblastic anemia, pernicious anemia, Crohn's disease, inflammatory bowel diseases, type 2 diabetes, cardiovascular diseases, stroke, dementia, cognitive impairment and osteoporosis; comprising determining if a subject has an impaired intestinal uptake of vitamin B12 by the method as defined by the second aspect of the invention.

In another aspect the present invention relates to a method for treating a subject with an impaired intestinal uptake of vitamin B12 comprising administering to the subject a vitamin B12 supplement; wherein the subject has been identified as having an impaired intestinal uptake of vitamin B12 by the method as defined by the second aspect of the invention.

In a further aspect the present invention provides a method for determining if a vitamin B12 deficiency in a subject is caused by an insufficient dietary uptake of vitamin B12 or an impaired intestinal absorption of vitamin B12; comprising performing the method as defined by the second aspect of the invention.

The method may further comprise the step of determining the level of vitamin B12, holotranscobalamine, methylmalonic acid and/or homocysteine in a sample isolated from the subject.

A subject determined to have an impaired intestinal absorption of vitamin B12 may be selected for vitamin B12 supplementation. Preferably the vitamin B12 supplementation is non oral vitamin B12 supplementation or oral vitamin B12 supplementation which is absorbed in the intestine with relatively high efficiency. For example cyanocobalamin and methylcobalamin are known to have a relatively high efficiency of absorption in the small intestine compared to other oral vitamin B12 supplements.

A subject determined to have an insufficient dietary uptake of vitamin B12 may be selected for oral vitamin B12 supplementation.

In a further aspect the present invention relates to a method for treating a subject with a vitamin B12 deficiency caused by an impaired intestinal uptake of vitamin B12 comprising administering to the subject a non-oral vitamin B12 supplement, or an oral vitamin B12 supplement which is absorbed from the intestine with relatively high efficiency; wherein the subject has been identified as having an impaired intestinal uptake of vitamin B12 by a method of the present invention.

In another aspect the present invention relates to a method for treating a subject with a vitamin B12 deficiency caused by an insufficient dietary uptake of vitamin B12 comprising administering to the subject an oral vitamin B12 supplement or a probiotic supplement comprising vitamin B12 producing bacteria; wherein the subject has been identified as having an insufficient dietary uptake of vitamin B12 by a method of the present invention.

In another aspect the present invention provides a vitamin B12 supplement for use in treating a subject with a vitamin B12 deficiency caused by an impaired intestinal uptake of vitamin B12, wherein the subject has been identified as having an impaired intestinal uptake of vitamin B12 by a method of the present invention. Preferably the vitamin B12 supplementation is non oral vitamin B12 supplementation or oral vitamin B12 supplementation which is absorbed from the intestine with relatively high efficiency.

The non-oral vitamin B12 supplement may be administered by, for example, parental, sub-lingual, or intra-nasal administration. Parenteral administration may include for example intra-muscular, sub-cutaneous, intradermal or intra-venous injection.

In another aspect the present invention provides an oral vitamin B12 supplement or a probiotic supplement comprising vitamin B12 producing bacteria for use in treating a subject with a vitamin B12 deficiency caused by an insufficient dietary uptake, wherein the subject has been identified as having an insufficient dietary uptake of vitamin B12 by a method of the present invention.

The oral vitamin B12 supplement may be a food product. The food product may comprise a probiotic supplement comprising vitamin B12 producing bacteria.

In a further aspect the present invention relates to the use of the cubam complex or a component thereof as a biomarker for the level of intestinal uptake of vitamin B12.

In another aspect the present invention provides the cubam complex or a component thereof for use in the diagnosis of a vitamin B12 deficiency.

In a further aspect the present invention provides an antibody which is capable of specifically binding to the cubam complex or a component thereof or a degradation product thereof.

In a further aspect the present invention relates to a use of an antibody as defined herein for determining levels of intestinal uptake of vitamin B12 in a subject, preferably wherein the level of intestinal uptake is determined by the method of the present invention.

In a further aspect the present invention relates to an antibody which is capable of specifically binding to the cubam complex or a component thereof for use is in diagnosing vitamin B12 deficiency associated with an impaired intestinal absorption.

The present invention thus provides a method for determining the level of intestinal uptake of vitamin B12 in a subject. The present invention also enables an improved rational provision of vitamin B12 supplementation for individuals suffering from a vitamin B12 deficiency associated with an impaired intestinal absorption or an insufficient dietary uptake of vitamin B12.

### DESCRIPTION OF THE DRAWINGS

FIG. 1—Relative amnionless level in rat serum increases with age. Somalogic multiplex technology (specific protein recognition by three-dimensional DNA sequences) was applied on rat serum from various ages. Among >100 regulated proteins, amnionless was found to be significantly increased from 18 m old corresponding to the onset of sarcopenia. Data are expressed as mean±S.E.M, n=10 per group. **p-value<0.01.

FIG. 2—Vitamin B12 serum concentration decreases with age. Total vitamin B12 concentration was measured in rat serum from various ages, using a competitive binding EIA. Vitamin B12 concentration is significantly decreased from 8 m old to 20 m old and stays stable from 20 m old to 24 m old. Data are expressed as mean±S.E.M, n=10 per group. *p-value<0.05; **p-value<0.01; ***p-value<0.001.

FIG. 3—Gastrocnemius muscle mass is decreases with age. Gastrocnemius muscles from rats at various ages were dissected out, weighed, and normalized to total body mass for each animal. A progressive decline is observed between 18 m old and 24 m old such that muscle weight from 24 m old animals represents half the muscle weight from 8 m old animals. Data are expressed as mean±S.E.M, n=10 per group. *p-value<0.05; **p-value<0.01; ′p-value<0.001.

FIG. 4—CD320 is expressed in rodent myotubes in vitro. Real time quantitative PCR was performed for CD320 on C2C12 cells during a time course of differentiation into myotubes (DMx indicates the number of days in differentiation medium). Data are presented normalized to the HPRT housekeeping gene and as a ratio compared to young animals. Data are expressed as mean±S.E.M, n=3 per group.

FIG. 5—A human amnionless nucleotide and amino acid sequence

FIG. 6—A human cubilin nucleotide and amino acid sequence

### DETAILED DESCRIPTION

The present method involves determining the level the cubam complex or a component thereof in an isolated sample from a subject.

Cubam Complex

The Cubam complex is the intrinsic factor (IF)-vitamin B12 receptor. It is a heterodimeric transmembrane receptor of two proteins, cubilin and amnionless (AMN) (Fyfe et al.; 2004; Blood: 103 (5)). Cubam is a multi-ligand receptor complex expressed in a variety of tissues, including ileum, kidney and yolk sac. In the proximal tubules of the kidney, cubam is involved in reabsorption of various proteins from the glomerularultrafiltrate (for example albumin, transferrin, apolipoprotein A-I and vitamin D-binding protein), thereby reducing proteinuria. In the ileum, the only known function of cubam is to facilitate uptake of dietary vitamin B12 in complex with its transport protein, IF (Pederson et al.; Traffic. May 2010; 11(5): 706-720).

Congenital inherited deficiencies in vitamin B12 may result from mutations in cubilin or amnionless causing Imerslund-Grasbeck syndrome.

A ‘component of the cubam complex’ may be either amnionless or cubilin.

Amnionless

Amnionless is ˜45 KDa type I transmembrane protein responsible for anchoring the cubam complex to the cell membrane and the endocytosis of the CBN-AMN-IF-B12 complex.

It contains two putative internalization signals of the FXNPXF type within its cytosolic domain which are active in terms of internalization of cubam and cubam ligands, possibly by engaging Disabled-2 (Dab2) and/or autosomal recessive hypercholesterolemia (ARH) (Pederson et al.; Traffic. May 2010; 11(5): 706-720).

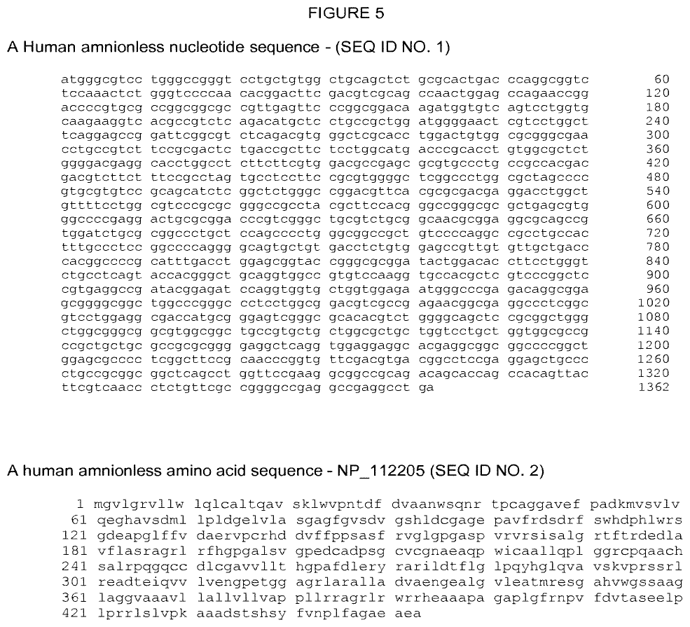

FIG. 5 shows a human amnionless nucleotide sequence corresponding to SEQ. ID. NO:1 and a human amnionless protein sequence (NCBI Reference Sequence: NP_112205) corresponding to SEQ. ID. NO:2.

It will be appreciated that the exact sequence of amnionless may vary between individuals and between species. These variants are all encompassed by the methods and uses of the present invention. For example, it could be envisaged that an aminoless sequence may have at least 65, 70, 75, 80, 85, 90, 95, 97 or 99% identity to the sequence (SEQ ID NO: 2) shown in FIG. 5.

Cubilin

Cubilin is ˜460 kDa glycosylated extra-cellular protein with a ligand binding domain for the intrinsic factor (Kozyraki et al.; Blood. 1998 May 15; 91(10):3593-600). The cubilin precursor protein undergoes proteolytic processing by the trans-Golgi proteinase furin to remove the 23 N-terminal amino acids.

Cubilin is composed of a short N-terminal region followed by eight epidermal growth factor (EGF)-like repeats and 27 contiguous CUB domains. Two distinct ligand binding regions have been identified. Region 1 (˜71 kDa), includes the 113-residue N terminus along with the eight epidermal growth factor (EGF)-like repeats and CUB domains 1 and 2; Region 2 (˜37 kDa) includes CUB domains 6-8 and binds both intrinsic factor-cobalamin (vitamin B(12); Cbl) (I F-Cbl) and albumin (Yammani et al.; J Biol Chem. 2001 Nov. 30; 276(48):44777-84). The cubilin protein includes no identifiable transmembrane region or classical signals for endocytosis.

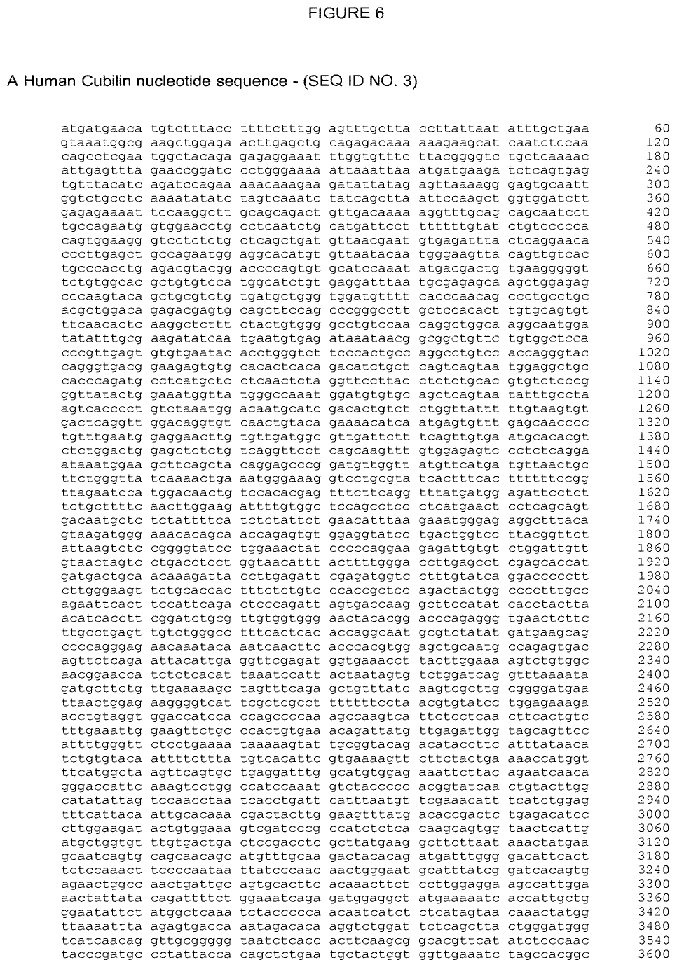

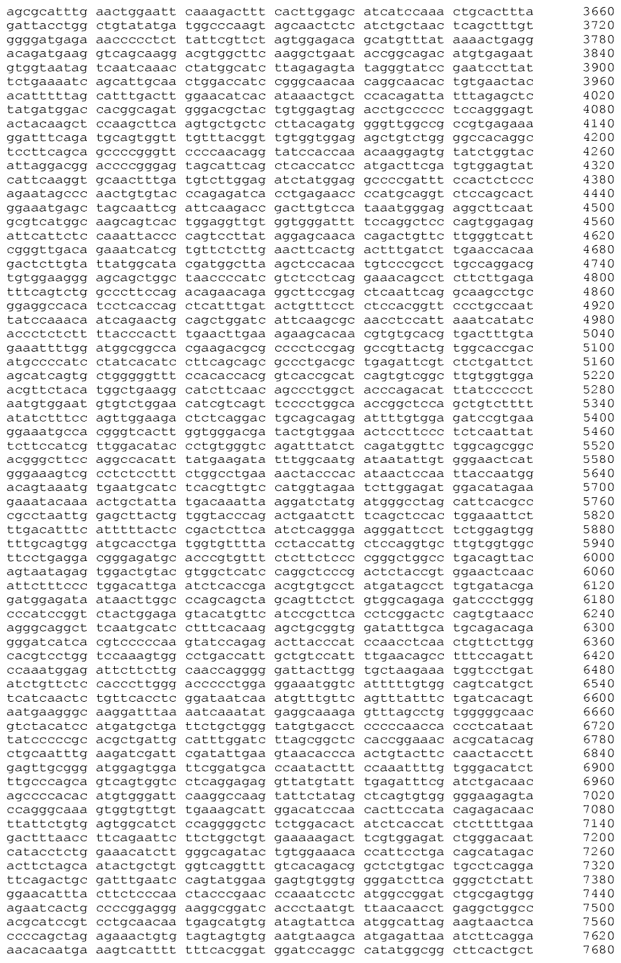

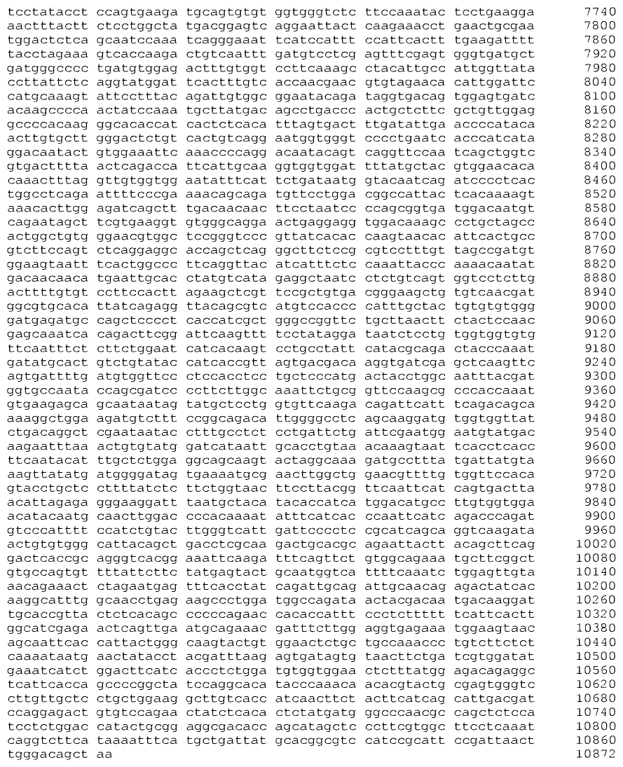

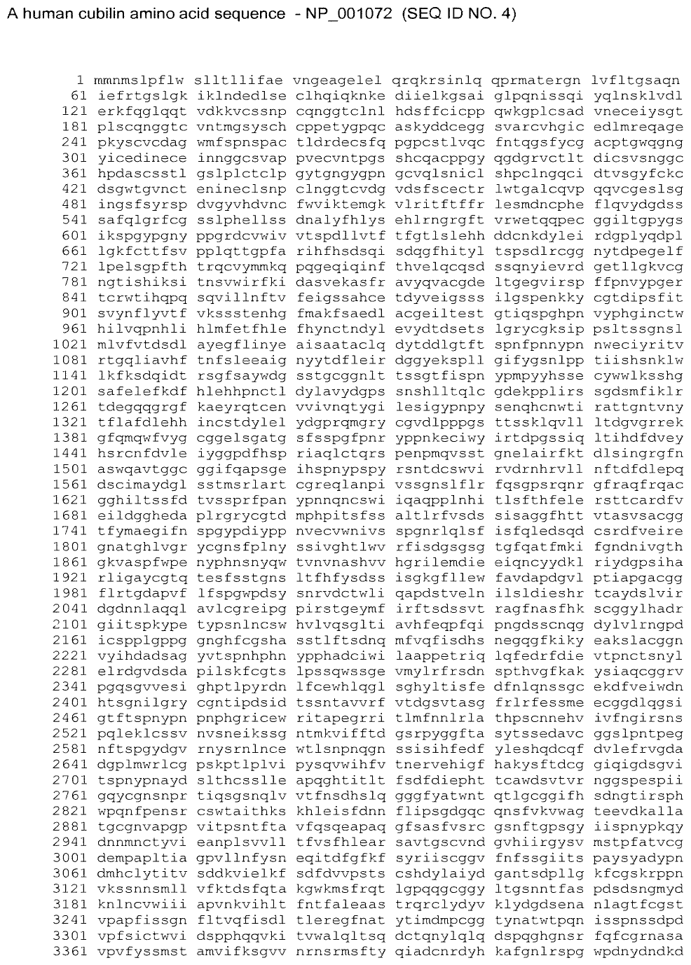

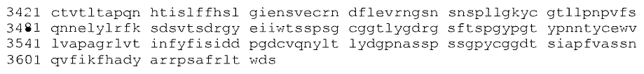

FIG. 6 shows a human cubilin nucleotide sequence corresponding to SEQ ID NO:3 and a human cubilin precursor protein sequence (NCBI Reference Sequence: NP_001072) corresponding to SEQ ID NO:4.

Canis familiaris cubilin mRNA and protein sequences are provided by NM_001003148.1 and NP_001003148.1, respectively.

It will be appreciated that the exact sequence of cubulin may vary between individuals and between species. These variants are all encompassed by the methods and uses of the present invention. For example, it could be envisaged that a cubulin sequence may have at least 65, 70, 75, 85, 90, 95, 97 or 99% identity to the amino acid sequence (SEQ ID NO:4) shown in FIG. 6.

The present method may involve detecting the cubam complex or a component thereof. The component of the cubam complex may be the full length mature amnionless or cubilin peptide, or a degradation product thereof.

‘Degradation product’ may be a peptide which is at least 1, 2, 5, 10, 20, 30, 40, 50, 60, 70, 80, 90 or 95, 98, or 99% of the full length amnionless or cubilin peptide.

In a further aspect the present invention provides the use of the cubam complex or a component thereof or a degradation product thereof, as a biomarker for the level of intestinal uptake of vitamin B12.

Vitamin B12

Vitamin B12 (also referred to as cobalamin) is a class of cobalt-containing hydrosoluble vitamins which cannot be synthetized by the human body and has to be taken up from food or synthesized by the gut microbiota.

Cobalamin may refer to several chemical forms of vitamin B12, depending on the upper axial ligand of the cobalt ion. These are:

Cyanocobalamin (R=—CN)

Hydroxocobalamin (R=—OH)

Methylcobalamin (R=—CH3), and

Adenosylcobalamin (R=-Ado).

Thus vitamin B12 comprises a class of chemically related compounds (vitamers), all of which have vitamin activity. It contains the biochemically rare element cobalt sitting in the centre of a planar tetra-pyrrole ring called a Corrin ring. Biosynthesis of the basic structure of the vitamin is accomplished only by bacteria (which usually produce hydroxocobalamin), but conversion between different forms of the vitamin can be accomplished in the human body.

The vitamin B12 pool in the human body is composed of several forms: cyanocobalamin, which is inactive and requires conversion for activity, and methylcobalamin and adenosylcobalamin, which are the metabolically active derivatives of vitamin B12.

After unloading from food matrices in the stomach, vitamin B12 binds to the intrinsic factor (IF) in the small intestine where it is absorbed through the cubam complex. Within the enterocytes, the CBN-AMN-IF-B12 complex is degraded in lysosomes through poorly characterized mechanisms, and free vitamin B12 exits the lysosome via the LMBD1 protein. Vitamin B12 is subsequently transported across the cytoplasm and the basolateral membrane of the enterocyte, and enters the bloodstream through a mechanism that requires at least in part the MRP1 transporter.

In the blood, vitamin B12 associates with two carrier proteins: haptocorrin, which carries approximately 80% of vitamin B12 and is not bioavailable to tissues, and transcobalamine, which carries approximately 20% of total vitamin B12 and is bioavailable to tissues by binding to the ubiquitous CD320 receptor, or to the asialoglycoprotein receptor complex in liver, which is the main site of vitamin B12 storage in the body.

Vitamin B12 is required for DNA synthesis and regulation, fatty acid metabolism and amino acid metabolism.

Level of Intestinal Uptake

‘Level of intestinal uptake’ refers to the amount of vitamin B12 from the dietary uptake which is absorbed into the blood via the intestine in a subject. As such, the level of intestinal uptake may represent the efficiency of intestinal uptake of vitamin B12 from the diet in a subject.

‘Impaired intestinal uptake’ may refer to an inefficient or inadequate uptake of dietary vitamin B12 in the intestine. For example, it may refer to a reduced intestinal uptake of vitamin B12.

Preferably, a subject ‘has an impaired intestinal uptake of vitamin B12’ if they do not absorb the required amount of vitamin B12 from a sufficient nutritional uptake of vitamin B12.

As such, an impaired intestinal uptake of vitamin B12 may contribute to, or be causative of, a vitamin B12 deficiency.

Levels of intestinal uptake of vitamin B12 may be compared to a reference level from a control cohort. The control subjects may be selected from a cohort which have been diagnosed with an impaired intestinal uptake of vitamin B12 and a cohort wherein the subjects have been predetermined not to have an impaired intestinal uptake of vitamin B12.

Vitamin B12Deficiency

The Recommended dietary allowance (RDA) of US adults was set at 2.4 pg per day by the Institute of Medicine, based on an average absorption from food of—50% (National Academy of Sciences, Institute of Medicine (2000); Dietary Reference Intakes for Thiamin, Riboflavin, Niacin, Vitamin B6, Folate, Vitamin B12, Pantothenic Acid, Biotin and Choline, Chapter 9, pp 306-56). It was noted that the daily requirement varies with body size.

The likelihood of vitamin B12 deficiency in humans may be defined according to the serum vitamin B12 level as follows: <148 picomols/L (<200 picograms/mL) indicates probable deficiency, 148 to 258 picomols/L (201 to 350 picograms/mL) indicates possible deficiency and >258 picomols/L (>350 picograms/mL) indicates that deficiency is unlikely (BMJ, Best Practice, http://bestpractice.bmj.com/best-practice/monograph/822/basics.html). However, because of the lack of a gold standard for determining vitamin B12 levels and related complications regarding active and inactive vitamin B12, assays of serum vitamin B12 are often combined with further biochemical assays or clinical assessment based on presenting symptoms, in order to diagnose vitamin B12 deficiency.

Additional assays which may be performed to give a further indication of a vitamin B12 deficiency include determining the level of, holotranscobalamine, methylmalonic acid and/or homocysteine in a sample isolated from the subject.

Holotranscobalamin refers to vitamin B12 bound to its bioactive serum transporter transcobalamine 11. Holotranscobalamin levels may be determined using commercial available assays (e.g., ELISA assays). Low levels of holotranscobalamin are associated with a potential vitamin B12 deficiency.

Methyl-malonic acid (MMA) accumulates with low activity of the vitamin B12-dependent enzyme methylmalonylCoA mutase. As such high levels of MMA are associated with vitamin B12 deficiency.

Homocysteine accumulates with low activity of the vitamin B12-dependent enzyme methionine synthase. High levels of homocysteine are associated with vitamin B12 deficiency. However assays of homocysteine levels can be confounded by folate deficiency.

The main syndrome of vitamin B12 deficiency is Biermer's disease (pernicious anemia). This is characterised by anemia with bone marrow promegaloblastosis (megaloblastic anemia), gastrointestinal and neurological symptoms.

Vitamin B12 deficiency can also cause symptoms of mania and psychosis, fatigue, memory impairment, irritability, depression and personality changes. In infants symptoms include irritability, failure to thrive, apathy, anorexia, and developmental regression.

Vitamin B12 deficiency is associated with a number of conditions and diseases. As such the level of intestinal uptake of vitamin B12 determined by the present method may be associated with intrinsic factor deficiency, Imerslund-Grasbeck syndrome, megaloblastic anemia, pernicious anemia, Crohn's disease, inflammatory bowel diseases, type 2 diabetes, cardiovascular diseases, stroke, dementia, cognitive impairment, a gastric bypass of part of the small intestine or osteoporosis, for example.

Age-Related Vitamin B12 Deficiency

Vitamin B12 deficiency in the elderly is well recognised (Hughes et al.; Ann Clin Biochem (2013); 50(4); 315-29) and low vitamin B12 has been linked to an increased risk of a number of age-related diseases, including cardiovascular disease, cognitive dysfunction, dementia and osteoporosis.

The impaired intestinal uptake of vitamin B12 determined by the present method may be associated with age-related vitamin B12 deficiency.

Sacropenia or Frailty

Age-related loss of muscle mass and function is inevitable in all individuals; however its progression largely depends on genetic and environmental factors such as physical activity or nutritional intake.

Sarcopenia has been defined as the point where the age-related loss of muscle mass and function gets debilitating and impacts quality of life. In contrast, frailty is another classification of age-related muscle dysfunction which relies on muscle strength and functionality, but not muscle mass.

Sarcopenia and frailty are multi-factorial syndromes which associate with pathophysiological changes such as impaired neuro-muscular transition, altered excitation/contraction coupling, impaired regenerative capacity linked to stem cell exhaustion, defects of mitochondrial and energy metabolism in myofibers, and finally marbling of skeletal muscle with fat and fibrosis. The etiology of these syndromes is therefore complex and poorly understood, but low physical activity, hormonal decline in anabolic hormones (androgens, IGF-1) and malnutrition/nutritional deficiencies play an important role. The most widely recognized nutritional deficiency in sarcopenic or frail patients is low levels of vitamin D.

The level of intestinal uptake of vitamin B12 determined by the present method may be associated with sarcopenia and/or frailty.

The term ‘associated with’ is used herein to indicate that the present method may be used to determine the level of intestinal uptake of vitamin B12 in a subject suffering from, or a risk of, a disease associated with vitamin B12 deficiency (for example as provided above).

The present invention further provides a method for diagnosing a disease selected from a group of: intrinsic factor deficiency, Imerslund-Grasbeck syndrome, megaloblastic anemia, pernicious anemia, Crohn's disease, inflammatory bowel diseases, type 2 diabetes, cardiovascular diseases, stroke, dementia, cognitive impairment and osteoporosis; comprising determining if a subject has an impaired intestinal uptake of vitamin B12 by the present method.

Determining Vitamin B12 Deficiency Associated with Intestinal or Dietary Uptake

In a further aspect the present invention relates to a method for determining if a vitamin B12 deficiency in a subject is caused by an insufficient dietary uptake of vitamin B12 or an impaired intestinal absorption of vitamin B12, wherein the level of intestinal uptake of vitamin B12 is determined according to the present method.

A subject is determined to have a vitamin B12 deficiency which is caused by an impaired intestinal uptake of vitamin B12 if they are vitamin B12 deficient and are determined to have an impaired intestinal uptake of vitamin B12 by the present method.

A subject is determined to have a vitamin B12 deficiency which is caused by an insufficient dietary uptake of vitamin B12 if they are vitamin B12 deficient and are determined not to have an impaired intestinal uptake of vitamin B12 by the present method.

A subject determined to have an impaired intestinal absorption of vitamin B12 may be selected for non-oral vitamin B12 supplementation.

A subject determined to have an insufficient dietary uptake of vitamin B12 may be selected for oral vitamin B12 supplementation.

The method may further comprise the step of determining the level one or more additional markers of vitamin B12 deficiency which are known in the art. For example the method may comprise the step of determining the level of vitamin B12, holotranscobalamine, methylmalonic acid and/or homocysteine in a sample isolated from the subject.

Preferably, the additional measurements are performed using the same sample used to determine the level of a component on the cubam complex.

Vitamin B12 Supplementation

The present invention further provides a method for treating a subject with an impaired intestinal uptake of vitamin B12 comprising administering to the subject a vitamin B12 supplement, wherein the subject has been identified as having an impaired intestinal uptake of vitamin B12 by the present method.

Vitamin B12 supplements are available in forms including cyanocobalamin, hydroxocobalamin, methylcobalamin, and adenosylcobalamin and may be provided singly or in combination with other supplements. The two main routes of vitamin B12 supplementation are oral ingestion from food supplements or parenteral injections (mainly intra-muscular, occasionally sub-cutaneous).

Advantageously, subjects determined by the present method as having an impaired intestinal uptake of vitamin B12 are treated with vitamin B12 supplements which do not require intestinal absorption.

The present method for treating a subject with an impaired intestinal uptake of vitamin B12 may therefore involve treating a subject determined to have an impaired intestinal absorption of vitamin B12 with a non-oral vitamin B12 supplement.

The present invention further provides a method for treating a subject with a vitamin B12 deficiency caused by an impaired intestinal uptake of vitamin B12 comprising administering to the subject a non-oral vitamin B12 supplement, wherein the subject has been identified as having an impaired intestinal uptake of vitamin B12 by the present method.

Non-oral vitamin B12 supplements may include, for example, supplements for parenteral, intramuscular, subcutaneous, sub-lingual, transdermal or intra-nasal administration.

The present method may involve treating a subject determined to have an impaired intestinal absorption of vitamin B12 with an oral vitamin B12 supplement which is absorbed from the intestine with a relatively high efficiency. For example cyanocobalamin and methylcobalamin are known to have a relatively high efficiency of absorption from the small intestine compared to other oral vitamin B12 supplements.

The present invention further provides a method for treating a subject with a vitamin B12 deficiency caused by an insufficient dietary uptake of vitamin B12 comprising administering to the subject an oral vitamin B12 supplement or a probiotic supplement comprising vitamin B12 producing bacteria; wherein the subject has been identified as having an insufficient dietary uptake of vitamin B12 by the present method.

Oral supplementation typically involves giving 250 fag to 1 mg of vitamin B12 daily.

The present method may comprise administering a probiotic supplement comprising vitamin B12 producing bacteria to a subject determined to have an insufficient dietary uptake of vitamin B12.

The probiotic supplement can include any probiotic microorganisms which beneficially affects the host subject by improving its intestinal microbial balance to enhance vitamin B12 uptake. The probiotic microorganism may be selected from the group comprising of Bifidobacterium, Lactobacillus, Streptococcus, Enterococcus and Saccharomyces or mixtures thereof.

Certain probiotic microorganisms which are native components of the gut microbiota are known to produce vitamin B12, for example, lactic acid producing bacteria such as Lactobacillus delbrueckii subsp. bulgaricus (see Le Blanc et al.; J App. Micro.; 111(6); (2011)). Advantageously, the probiotic supplement can enhance existing microorganisms in the gut that produce vitamin B12 in situ.

The oral supplementation may be in the form of a food or beverage product. The food or beverage product may comprise a probiotic supplement comprising vitamin B12 producing bacteria or other probiotics which can enhance existing microorganisms in the gut that produce vitamin B12 in situ.

Typically, a physician will determine the actual dosage which will be most suitable for an individual subject and it will vary with the age, weight and response of the particular patient. The dosage is such that it is sufficient to provide required levels of active vitamin B12.

Subject

The subject may be, but is not limited to, mammals such as bovine, canine, caprine, cervine, equine, feline, human, ovine, porcine and primates. Preferably the subject is a human. In various embodiments, the subject may have, or be suspected of or at risk of, a vitamin B12 deficiency.

The subject may suffer from a disease selected from a group of: intrinsic factor deficiency, Imerslund-Grasbeck syndrome, megaloblastic anemia, pernicious anemia, Crohn's disease, inflammatory bowel diseases, type 2 diabetes, cardiovascular diseases, stroke, dementia, cognitive impairment, bypass surgery of part of the digestive tract and osteoporosis.

The subject may be an elderly individual suffering from, or at risk of, sarcopenia and/or frailty.

Vitamin B12 supplementation is commonly used as part of a sports nutritional supplementation programme. The present methods may be used to determine the most efficiency method of vitamin B12 supplementation as part of a sports nutritional supplementation programme.

Sample

The present method involves determining the level of the cubam complex or a component thereof in a sample obtained from a subject. This may be referred to as the ‘test sample’. Thus the present method is typically practiced outside of the human or animal body, e.g. on a body fluid sample that was previously obtained from the subject to be tested.

The sample may, for example, be a serum, plasma or urine sample.

Preferably the sample is derived from blood, i.e. the sample comprises whole blood or a blood fraction. Most preferably the sample comprises blood plasma or serum.

Techniques for collecting blood samples and separating blood fractions are well known in the art. For instance, vena blood samples can be collected from patients using a needle and deposited into plastic tubes. The collection tubes may, for example, contain spray-coated silica and a polymer gel for serum separation. Serum can be separated by centrifugation at 1300 RCF for 10 min at room temperature and stored in small plastic tubes at −80° C.

Comparison to Reference Levels

The present method involves a comparison to reference levels. The term reference level is synonymous with ‘control level’ and broadly includes data that the skilled person would use to facilitate the accurate interpretation of technical data.

The level of a target in a test sample, for example the level of the cubam complex or a component thereof in a sample from the subject, may be compared to the respective level of the same target in one or more cohorts (populations/groups) of control subjects. The control subjects may be selected from a cohort which have been diagnosed with an impaired intestinal uptake of vitamin B12 and a cohort wherein the subjects have been predetermined not to an impaired intestinal uptake of vitamin B12.

The reference value for the level of the cubam complex or a component thereof is preferably measured using the same units used to characterize the level of the cubam complex or a component thereof in the test sample. Thus, if the level of the level of the cubam complex or a component thereof is an absolute value such as the units of the cubam complex or a component thereof in pmol/l (pM) the reference value is also based upon the units of the cubam complex or a component thereof in pmol/l (pM) in individuals in the general population or a selected control population of subjects.

Moreover, the reference value can be a single cut-off value, such as a median or mean. Reference values of the cubam complex or a component thereof in obtained body fluid samples, such as mean levels, median levels, or “cut-off” levels, may be established by assaying a large sample of individuals in the general population or the selected population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is incorporated herein by reference.

In is known in the art how to assign correct reference values as they will vary with gender, race, genetic heritage, health status or age, for example.

In particular, the present inventors have demonstrated that the level of the cubam complex or a component thereof varies with the age of the subject. As such the reference levels should be age-matched with the test sample.

The control or reference levels for the level of the cubam complex or a component thereof in a particular sample may be stored in a database and used in order to interpret the results of the method as performed on the subject.

The present method provides that a difference in the level of the cubam complex or a component thereof in the test sample compared to the reference level is indicative of the level of intestinal uptake of vitamin B12.

The present method may provide that a difference in the level of the cubam complex or a component thereof in the test sample compared to the reference level is indicative of an impaired intestinal uptake of vitamin B12.

For example, a 1.1, 1.5, 2, 3, 4, 5, 10, 50, 100 or 1000-fold difference between the levels determined in the test sample and the reference level may be indicative of an impaired level of intestinal uptake of vitamin B12.

In a particular embodiment, a higher level of the cubam complex or a component thereof in the test sample compared to the reference level may be indicative of a reduced level of intestinal uptake of vitamin B12. The level of the cubam complex or a component thereof may be 1.1, 1.5, 2, 3, 4, 5, 10, 50, 100 or 1000-fold greater in the test sample compared to the reference level.

In a preferred embodiment, the level of the cubam complex or a component thereof in a serum sample from a subject may be 1.1, 1.5, 2, 3, 4, 5, 10, 50, 100 or 1000-fold greater than the reference level.

Detection Method

The level of the cubam complex or a component thereof may be detected using a variety of suitable methods and techniques known in the art.

Typically the level of the cubam complex or a component thereof in the sample and the reference value are determined using the same analytical method.

The determination may involve the detection of a polypeptide or a degradation product thereof.

The step of determining the level of the cubam complex or a component thereof may involve the detection of the polypeptide using a technique such as flow cytometry, antibody-based arrays, enzyme linked immunosorbent assay (ELISA), non-antibody protein scaffolds (e.g. fibronectin scaffolds), radioimmuno-assay (RIA), western blotting, aptamers or mass spectrometry for example.

An ELISA may be performed according to general methods which are known in the art. For example, the ELISA may be a sandwich or competitive ELISA

A sandwich ELISA may comprise the following steps:
a surface (i.e. a microtitre plate well) is prepared to which a known quantity of capture antibody is bound;
any nonspecific binding sites on the surface are blocked;
the sample comprising the cubam complex or a component thereof is applied to the plate;
the plate is washed to remove unbound antigen;
a primary antibody which is capable of specifically binding the cubam complex or a component thereof is added, and binds to antigen;
an enzyme-linked secondary antibody is applied as a detection antibody that bind specifically to the antibody Fc region;
the plate is washed to remove the unbound antibody-enzyme conjugates, before a chemical is added to be converted by the enzyme into a colour or fluorescent or electrochemical signal;
the absorbency or fluorescence or electrochemical signal of the plate wells is measured to determine the presence and quantity of antigen.

A competitive ELISA may comprise the following steps:
a labelled antibody which specifically binds the cubam complex or a component thereof is incubated in the presence of a sample comprising the cubam complex or a component thereof;
the bound antibody/antigen complexes are then added to an antigen-coated well;
the plate is washed, so unbound antibody is removed;
a secondary antibody, specific to the primary antibody, and coupled to an enzyme is added;
a substrate for the enzyme is added, and the reaction of the enzyme with its substrate elicits a chromogenic or fluorescent signal;
the reaction is stopped to prevent eventual saturation of the signal.

Various enzyme-substrate labels are available, e.g. as disclosed in U.S. Pat. No. 4,275,149. The enzyme generally catalyses a chemical alteration of the chromogenic substrate that can be detected. For example, the enzyme may catalyse a colour change in a substrate, or may alter the fluorescence or chemiluminescence of the substrate. Examples of enzymatic labels include peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are well known.

A detection using aptamers may comprise the following steps:
Aptamers that specifically recognize the cubam complex or a component may be synthesized using standard nucleic acid synthesis techniques or selected from a large random sequence pool, for example using the Systematic Evolution of Ligands by Exponential Enrichment (SELEX) technique;
Aptamers are mixed with the samples so that aptamer-protein complex are formed;
Non-specific complexes are separated;
Bound aptamers are removed from their target proteins;
Aptamers are collected and measured, for example using microarrays or mass spectrometry techniques.

Aptamers can be single strand DNA or RNA sequences that fold in a unique 3D structure having a combination of stems, loops, quadruplexes, pseudoknots, bulges, or hairpins. The molecular recognition of aptamers results from intermolecular interactions such as the stacking of aromatic rings, electrostatic and van der Waals interactions, or hydrogen bonding with a target compound. In addition, the specific interaction between an aptamer and its target is complemented through an induced fit mechanism, which requires the aptamer to adopt a unique folded structure to its target. Aptamers can be modified to be linked with labeling molecules such as dyes, or immobilized on the surface of beads or substrates for different applications.

Aptamers can be paired with nanotechnology, microarray, microfluidics, mass spectrometry and other technologies for quantification in a given sample.

Homology

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbial. Lett. (1999) 174: 247-50; FEMS Microbial. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

Antibody

The present invention provides an antibody which is capable of specifically binding to the cubam complex or a component thereof, or a degradation product thereof.

The antibody may be capable of specifically binding to amnionless or a degradation product thereof.

The term “antibody” is used herein to relate to an antibody or a functional fragment thereof. By functional fragment, it is meant any portion of an antibody which retains the ability to bind to the same antigen target as the parental antibody—i.e retains the ability to bind to the cubam complex or a component thereof or a degradation product thereof.

The antibody may be a chimeric antibody. Chimeric antibodies may be produced by transplanting antibody variable domains from one species (for example, a mouse) onto antibody constant domains from another species (for example a human).

The antibody may be a full-length, classical antibody. For example the antibody may be an IgG, IgM or IgA molecule.

The antibody may be a functional antibody fragment. Specific antibody fragments include, but are not limited to, (i) the Fab fragment consisting of VL, VH, CL and CHI domains, (ii) the Fd fragment consisting of the VH and CHI domains, (iii) the Fv fragment consisting of the VL and VH domains of a single antibody, (iv) the dAb fragment, which consists of a single variable domain, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site, (viii) bispecific single chain Fv dimers, and (ix) “diabodies” or “triabodies”, multivalent or multispecific fragments constructed by gene fusion.

The antibody fragments may be modified. For example, the molecules may be stabilized by the incorporation of disulphide bridges linking the VH and VL domains.

The antibody described herein may be a multispecific antibody, and notably a bispecific antibody, also sometimes referred to as “diabodies”. These are antibodies that bind to two (or more) different antigens. Diabodies can be manufactured in a variety of ways known in the art, e.g., prepared chemically or from hybrid hybridomas. The antibody may be a minibody. Minibodies are minimized antibody-like proteins comprising a scFv joined to a CH3 domain. In some cases, the scFv can be joined to the Fc region, and may include some or all of the hinge region.

The antibody may be a domain antibody (also referred to as a single-domain antibody or nanobody). This is an antibody fragment containing a single monomeric single variable antibody domain. Examples of single-domain antibodies include, but are not limited to, VHH fragments originally found in camelids and VNAR fragments originally found in cartilaginous fishes. Single-domain antibodies may also be generated by splitting the dimeric variable domains from common IgG molecules into monomers.

The antibody may be a synthetic antibody (also referred to as an antibody mimetic). Antibody mimetics include, but are not limited to, Affibodies, DARPins, Anticalins, Avimers, Versabodies and Duocalins.

The invention will now be further described by way of Examples, which are meant to serve to assist one of ordinary skill in the art in carrying out the invention and are not intended in any way to limit the scope of the invention.

### EXAMPLES

### Example 1—Increased Levels of Serum Amnionless Correlate with Age

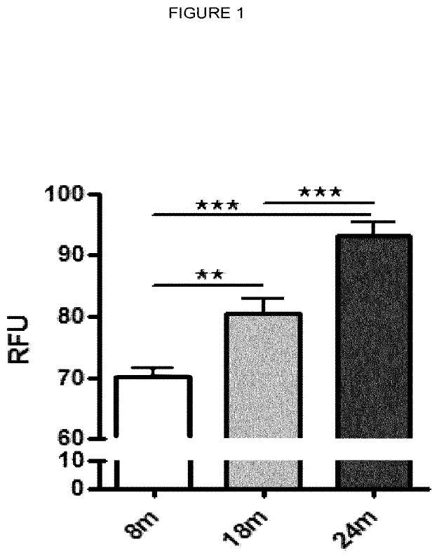

Using Somalogic technology to identify age-related changes in rat serum proteins, the inventors have found that the rat serum levels of amnionless significantly increased with age (FIG. 1).

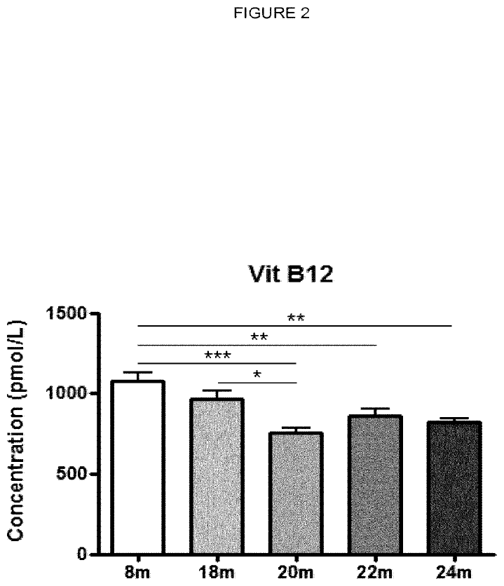

Levels of cobalamin in rat serum were measured using a competitive binding immunoluminescent enzymatic assay, which demonstrated a decrease in total vitamin B12 levels with age (FIG. 2). This was consistent with the increasing levels of amnionless determined in the serum. As it is known that vitamin B12 hepatic pools can provide vitamin B12 to the rest of the body for several weeks upon low vitamin B12 intake or absorption, the fact that amnionless levels increased at 18 months of age before the decrease of the circulating levels vitamin B12 occurring at 20 months indicates that the amnionless changes could be causal for the age-related vitamin B12 deficiency.

### Example 2—Changes in Amnionless Levels are Associated with Muscle Mass Decline

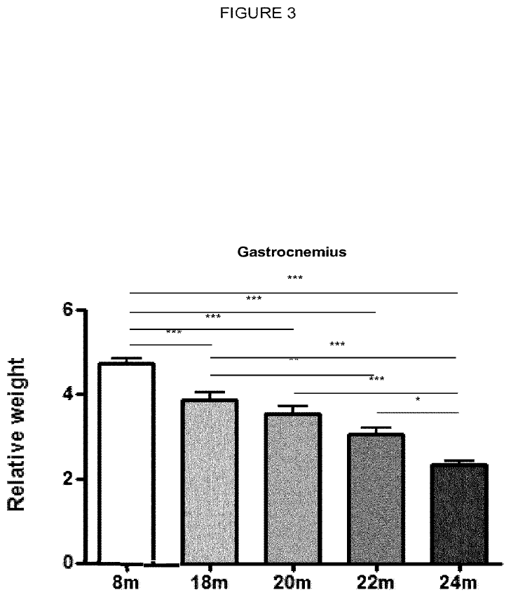

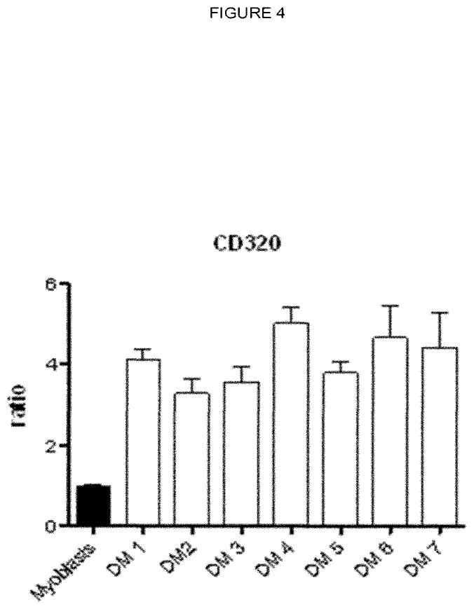

It was further observed that the amnionless and vitamin B12 changes in rat serum occur from 18 months of age onwards, corresponding to the onset of muscle mass decline observed in hind-limb muscles of aging rats. This demonstrated for Gastrocnemius muscle in FIG. 3. Preliminary in vitro experiments demonstrated that the tissue VitB12 receptor CD320 is expressed on differentiated muscle cells but not in their proliferating progenitors (FIG. 4), suggesting that vitamin B12 can enter skeletal muscle and influence muscle physiology.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

The invention is claimed as follows:

1: A method of treating a subject with an impaired intestinal uptake of vitamin B12, the method comprising administering to the subject a vitamin B12 supplement, wherein the subject has been identified as having an impaired intestinal uptake of vitamin B12.

2: The method of claim 1 comprising:
determining a level of a cubam complex or component thereof in an isolated sample from the subject, wherein the cubam complex or component thereof comprises amnionless and/or cubulin or a degradation product of thereof; and
comparing the level of the cubam complex or component thereof to reference levels,
wherein the level of the cubam complex or component thereof compared to the reference levels is indicative of the impaired intestinal uptake of vitamin B12.

3: The method of claim 1, wherein the impaired intestinal uptake of vitamin B12 is indicative of a vitamin B12 deficiency.

4: The method of claim 1, wherein the impaired intestinal uptake of vitamin B12 is associated with a disease or condition selected from a group of instrinsic factor deficiency, Imerslund-Grasbeck syndrome, megalobastic anemia, pernicious anemia, Crohn's disease, inflammatory bowel diseases, type 2 diabetes, cardiovascular diseases, stroke, dementia, cognitive impairment, bypass surgery of part of the digestive tract, osteoporosis, and combinations thereof.

5: The method of claim 1, wherein the impaired intestinal uptake of vitamin B12 is associated with age-related deficiency in vitamin B12.

6: The method of claim 1, wherein the impaired intestinal uptake of vitamin B12 is associated with sarcopenia and/or frailty.

7: A method of treating a subject with a vitamin B12 deficiency caused by an impaired intestinal uptake of vitamin B12, the method comprising administering to the subject a non-oral vitamin B12 supplement, wherein the subject has been identified as having the impaired intestinal uptake of vitamin B12.

8: The method of claim 7 comprising determining if the vitamin B12 deficiency in the subject is caused by an insufficient dietary uptake of vitamin B12 or the impaired intestinal absorption of vitamin B12.

9: The method of claim 7 comprising:
determining a level of a cubam complex or a component thereof in an isolated sample from the subject, wherein the cubam complex or component thereof comprises amnionless and/or cubulin or a degradation product of thereof; and
comparing the level of the cubam complex or component thereof to reference levels,
wherein the level of the cubam complex or component thereof compared to the reference levels is indicative of the impaired intestinal uptake of vitamin B12.

10: The method of claim 7, wherein the impaired intestinal uptake of vitamin B12 is indicative of the vitamin B12 deficiency.

11: The method of claim 7, wherein the impaired intestinal uptake of vitamin B12 is associated with a disease or condition selected from a group of: instrinsic factor deficiency, Imerslund-Grasbeck syndrome, megalobastic anemia, pernicious anemia, Crohn's disease, inflammatory bowel diseases, type 2 diabetes, cardiovascular diseases, stroke, dementia, cognitive impairment, bypass surgery of part of the digestive tract and osteoporosis.

12: The method of claim 7, wherein the impaired intestinal uptake of vitamin B12 is associated with age-related deficiency in vitamin B12.

13: The method of claim 7, wherein the impaired intestinal uptake of vitamin B12 is associated with sarcopenia and/or frailty.

14: The method of claim 7, wherein the non-oral vitamin B12 supplement is administered by parental, sub-lingual, sub-cutaneous, transdermal or intra-nasal administration.

15: A method of treating a subject with a vitamin B12 deficiency caused by an insufficient dietary uptake of vitamin B12, the method comprising administering to the subject an oral vitamin B12 supplement, wherein the subject has been identified as having the insufficient dietary uptake of vitamin B12.

16: The method of claim 15 comprising determining if the vitamin B12 deficiency in the subject is caused by an insufficient dietary uptake of vitamin B12 or the impaired intestinal absorption of vitamin B12.

17: The method of claim 16 comprising:
determining a level of a cubam complex or component thereof in an isolated sample from the subject, wherein the cubam complex or component thereof comprises amnionless and/or cubulin or a degradation product of thereof; and
comparing the level of the cubam complex or component thereof to reference levels,
wherein the level of the cubam complex or component thereof compared to the reference levels is indicative of the impaired intestinal uptake of vitamin B12.

18: The method of claim 15, wherein the oral vitamin B12 supplement is a food product or food product comprising a probiotic supplement comprising vitamin B12 producing bacteria.

19: A method of vitamin B12 supplementation in a subject, the method comprising:
determining a level of a cubam complex or component thereof in a sample from the subject;
comparing the level of the cubam complex or component thereof from the sample to reference levels; and
administering to the subject a composition comprising one or more of a probiotic supplement, a vitamin B12 producing bacteria, a non-oral vitamin B12 supplement, and a high absorption efficiency vitamin B12 supplement, if a higher level of the cubam complex or component thereof is detected in the sample from the subject compared to the reference levels.
